(19) United States
(12) Patent Application Publication
(10) Pub. No.: US 2009/0247846 A1
(43) Pub. Date: Oct. 1, 2009

(54) PULSE OXIMETER WITH REDUCED CROSS TALK EFFECTS

(75) Inventor: Borje Rantala, Helsinki (FI)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/060,489

(22) Filed: Apr. 1, 2008

(51) Int. Cl. A61B 5/00

(52) U.S. Cl. 600/323

(57) ABSTRACT

A method of reducing the effect of cross talk in a pulse oximeter is disclosed herein. The method includes: setting an initial pulse width and pulse rate for an LED drive signal and measuring a cross talk generated within the pulse oximeter. Based on the measured cross talk, the pulse width of the LED drive signal is minimized. The pulse width is minimized based on a relationship between the cross talk and a threshold level.

### CROSS-REFERENCE TO RELATED PATENT

This application is related to commonly assigned U.S. Pat. No. 6,963,767 B2, granted on Nov. 8, 2005 to Rantala, et al. and entitled “Pulse Oximeter”, which is herein incorporated by reference in its entirety.

### FIELD OF THE INVENTION

The invention relates generally to pulse oximeter, and more particularly to, a method and system for reducing effects of cross talks in a pulse oximeter.

### BACKGROUND OF THE INVENTION

Pulse oximetry is at present the standard of care for the continuous monitoring of arterial oxygen saturation (SpO2). Pulse oximeters provide instantaneous in-vivo measurements of arterial oxygenation, and thereby provide early warning of arterial hypoxemia, for example. A conventional pulse oximeter comprises a probe including at least two LED emitters and a photo detector to detect the lights. The probe is connected to a patient's finger tip or to the ear lobe. The light from the emitters is passed through the tissue and the photo detector detects the light and, based on the transmitted and received light, light absorption by the tissue is evaluated.

The accuracy of the pulse oximeter is determined by several factors. An important factor affecting the accuracy of a pulse oximeter is the direct electrical crosstalk between the circuitry driving the LEDs and the circuitry receiving the signal from the photo detector. Due to crosstalk of this type, non-optical signal components may superimpose on the signal received and thus cause erroneous oxygen saturation readings. This problem does not exist with conventional pulse oximeters using wide pulses, where power consumption is not a major concern.

But in the case of portable oximeters, especially battery operated oximeters, the power consumption is a major concern and the pulse width cannot be increased beyond a certain level. Lower power consumption calls for narrower pulses for driving the LEDs, the narrower pulses being more vulnerable to this type of crosstalk. The problem is further aggravated if the tissue of the patient is thicker than normal, whereby the signal received from the photo diode detector is weaker than normal.

Further the cross talk generated within a pulse oximeter depends on topology of the circuitry driving the LEDs and the circuitry receiving detect signal from the photodiode detector and the topology of the cable that connects the detector with the circuitry receiving detect signal or the cable that connects or feeds the drive signal for driving the LED emitters in the pulse oximeter. The cross talk generated may vary based on the configuration of the cable or the probe used. Since the probes are manufactured by different manufactures, the allowable cross talk limit, noise etc may vary. Thus even though many of the pulse oximeters today are configured to accept different types of probes, manufactured by different manufactures, special attention needs to be given in addressing effects of cross talks generated by these probes.

Thus there exists a need to provide an improved low power pulse oximeter, wherein the effect of cross talk is configured to be a minimum.

### SUMMARY OF THE INVENTION

The above-mentioned shortcomings, disadvantages and problems are addressed herein which will be understood by reading and understanding the following specification.

One embodiment of the present invention provides a method for reducing effects of cross talk in a pulse oximeter. The method includes: setting an initial pulse width and pulse rate for an LED drive signal; measuring a cross talk generated within the pulse oximeter; and minimizing the pulse width of the LED drive signal with reference to the cross talk.

In another embodiment, a pulse oximetry method is described. The method includes: setting initial pulse width and pulse rate for an LED drive signal; obtaining cross talk generated within the pulse oximeter; comparing value of the cross talk with a threshold value; adjusting the duty cycle of the LED drive signal upon detecting cross talk value as below the threshold value; and initiating the measurement of oxygenation once the pulse width is adjusted to a desirable value.

In yet another embodiment, a method of monitoring arterial oxygen saturation (SpO2) using a plurality of light emitters and at least one detector is disclosed. The method includes: initiating monitoring of the oxygenation using an emitter drive signal having preset initial characteristics; obtaining cross talk generated within the pulse oximeter; determining an error value generated in the measurement of oxygenation due to the cross talk; comparing the error value with a threshold value; adjusting the duty cycle of the emitter drive signal until the error value is below the threshold value; and identifying the arterial oxygen saturation (SpO2).

In yet another embodiment, a method of configuring a pulse oximeter to adapt multiple probes is disclosed. The method includes: selecting at least one probe from a plurality of probes; activating the selected probe by a drive signal having preset initial characteristics; accessing value of cross talk generated within the pulse oximeter in relation to the selected probe; obtaining a threshold value of permissible cross talk for the selected probe; and minimizing duty cycle of the drive signal in reference to the cross talk generated due to the probe, the duty cycle being minimized until the cross talk reaches the threshold value.

In yet another embodiment, a pulse oximeter is disclosed. The system comprises: at least one probe including at least two emitters configured to emit radiations in different wavelengths and at least one detector to receive radiations; a drive unit for providing drive signal to the emitters, the drive signal configured to have preset initial characteristics; a cross talk identifier configured to identify electrical cross talk synchronous with the drive signal, generated within the pulse oximeter; and a processor configured to adjust duty cycle of the drive signal in reference to the cross talk.

Various other features, objects, and advantages of the invention will be made apparent to those skilled in the art from the accompanying drawings and detailed description thereof.

### BRIEF DESCRIPTION OF THE DRAWINGS

FIG. 1 is a flowchart illustrating a method of reducing effects of cross talk in a pulse oximeter as described in an embodiment of the invention;

FIG. 2 is a flowchart illustrating a pulse oximetry method as described in an exemplary embodiment of the invention;

FIG. 3 is a flowchart illustrating a pulse oximetry method as described in another exemplary embodiment of the invention;

FIG. 4 is a flowchart illustrating a method of configuring a pulse oximetry system to adapt multiple probes as described in an embodiment of the invention; and

FIG. 5 is a detailed block diagram of a pulse oximeter as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

Various embodiments of the present invention provide a method of reducing the effects of cross talk in a pulse oximeter. The invention addresses the electrical cross talks generated within the pulse oximeter.

In an embodiment, the invention facilitates a method of controlling the power consumption in a pulse oximeter. The power consumption is minimal, when the pulse width of the drive signal is minimum. However if the pulse width is minimal, the cross talk will adversely affect the accuracy of oxygenation measurement. Thus an optimum value of duty cycle needs to be derived where the effect of cross talk is minimum on the oxygenation measurement, but still the pulse oximeter is power efficient.

In an embodiment, the invention suggests a method of varying duty cycle of a drive signal in a pulse oximeter system in real time, even while the system monitors the oxygenation.

In an embodiment, with reference to the cross talk, the duty cycle of the detector signal is modified to avoid the effect of cross talk in the oxygenation measurement.

The term “cross talk” referred to in the specification refers to electrical cross talk generated within the pulse oximeter due to the various power coupling such as resistive, inductive, capacitive etc. The cross talk may occur due to the various cables involved in the pulse oximetry system. Mainly the cross talk due to the cable that carries drive signal from a drive unit to the LEDs in the probe and another cable that carries the detecting signal from the detector located inside the probe to an amplifier.

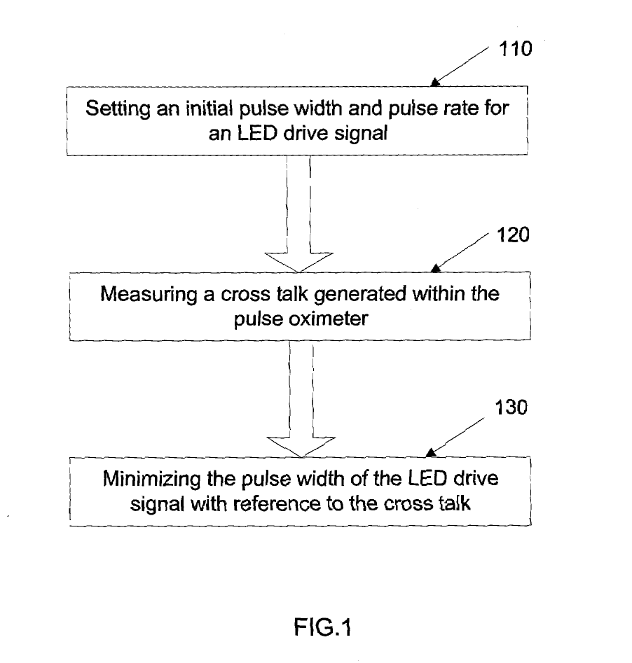

FIG. 1 is a flowchart illustrating a method of reducing effect of cross talk in a pulse oximeter as described in an embodiment of the invention. At step 110, an LED drive signal in a pulse oximeter is defined with an initial pulse width and pulse rate. A pulse oximeter is provided with a probe including emitters and photo detectors. The emitters are configured to emit light that are passed through a tissue and the photo detector is configured to detect the light that comes out of the tissue. The LEDs are driven by a drive signal. The drive signal triggers the LEDs and in an example, the LEDS need to be switched on at a frequency of about 100 to 500 pulses per millisecond. For portable pulse oximeters the frequency is higher. The drive signal is in the form of current pulses having varying magnitude. Initial frequency of repetition and pulse width or the duty cycle of the drive signal is set at a preset value, based on the application or the probes used.

At step 120, cross talk generated within the pulse oximeter is measured. Different mechanism such as using a leakage resistor, changing the amplitude of the drive signal in a fixed pattern etc may be used in measuring the cross talk. The cross talk measurement need not be limited to the examples mentioned; the system may use any technique for measuring the cross talk. In an embodiment, the electrical cross talk synchronous with the LED drive signal is measured. The electrical cross talk could be due to at least one of capacitive, resistive or inductive power coupling from the driving signal to the detect signal or in the drive signal due to the cable topology. The cross talk caused by resistive, captive or inductive power coupling may be measured together or separately. Further the cross talk could be measured before measurement of the oxygenation level, based on some standard values or could be measured during the arterial oxygen saturation (SpO2).

At step 130, the pulse width is minimized with reference to the cross talk. For each probe used in monitoring the oxygenation, there is a threshold value indicating the allowable cross talk level. The measured cross talk is compared with the threshold value and, based on the same, pulse width of the drive signal is minimized. The pulse width is increased until the cross talk remains within the threshold value. The reduction in pulse width will reduce the power consumption as well.

In an example, an error signal may be generated from the measured oxygenation signal. This error signal could be compared with a standard threshold value that indicates the allowable error limit and if the error value is within the threshold value, the pulse width may be minimized. By minimizing the pulse width, while monitoring the cross talk, will ensure a power efficient method with the cross talk having least impact on the measurement.

In an example, the pulse width of detect signal that is detected by the detector may be adjusted to reduce the impact of cross talks on the measurement of oxygenation. The pulse width of the detect signal is varied based on the cross talk. The pulse width may be increased to delay the sampling of the detect signal. This facilitates the cross talk to die off before sampling of the detect signal.

In an embodiment, based on the measured cross talk, the pulse rate or frequency of the drive signal may be modified or adjusted. This adjustment will also assist in reducing the impact of cross talk.

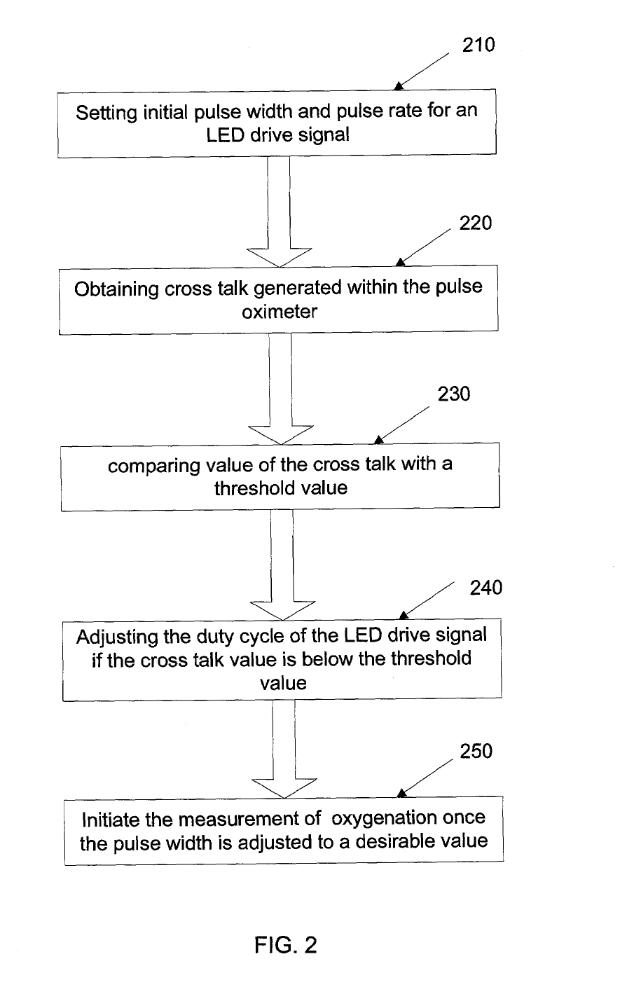

FIG. 2 is a flowchart illustrating a pulse oximetry method as described in an exemplary embodiment of the invention. Pulse oximetry method is used to record the level of oxygenation in the blood. At step 210, an initial pulse width or pulse rate is set for the LED drive signal. This may be done based on the application and/or based on the probes or topology of electrical components present in the pulse oximetry system. At step 220, cross talk generated within the pulse oximetry system is measured. The cross talk synchronous with the LED drive signal is measured. The cross talk may be measured using a leakage resistor or by amplitude modulating the drive signal. If the cross talk is determined prior to the oxygenation measurement, the cross talk may be measured based on a trail input. At step 230, the measured value of the cross talk is compared with a threshold value. The threshold value may be defined based on the application or may be provided in the specification of the probe itself. At step 240, the duty cycle or pulse width of the LED drive signal is adjusted. The pulse width of the drive signal may be adjusted by considering the cross talk. If the cross talk measured in the pulse oximeter is less than the threshold value, the pulse width of the drive signal may be modified. By reducing the pulse width, the power consumption is reduced. Also, based on the cross talk, the pulse width of the detect signal detected by the photo detector in the pulse oximeter, may be increased so that the cross talk can die off before sampling the detect signal. At step 250, the measurement of oxygenation in initiated. The measurement could be initiated once a desired pulse level is set. However the cross talk can be monitored in real time, even while measuring the oxygenation and the pulse width may be adjusted accordingly.

In an example, there could be multiple probes used with a pulse oximeter system. These multiple probes include different probes manufactured by different manufacturers. Thus, different probes may have different threshold allowable cross talk levels and based on the same pulse width of the corresponding drive signal may be adjusted.

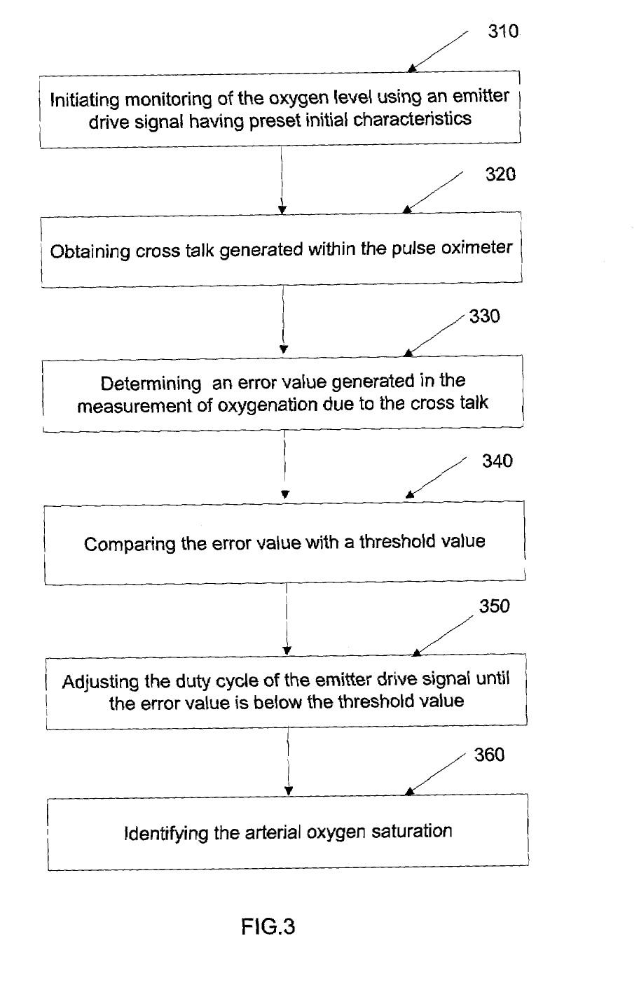

FIG. 3 is a flowchart illustrating a pulse oximetry method as described in another exemplary embodiment of the invention. At step 310, monitoring of the blood oxygenation level is initiated using an emitter drive signal having preset initial characteristics. The initial characteristics may be set based on the application and/or based on the probes or other electrical components present in the pulse oximetry system. The emitter drive signal with preset characteristics is configured to trigger the emitters and emit light. The emitted light is passed through a tissue, such as a finger tip or an ear lobe, and is detected by a photo detector. At step 320, the cross talk generated within the pulse oximeter is obtained. The electrical cross talk synchronous with the LED drive signal is measured. At step 330, an error value generated in the oxygenation measurement due to the cross talk is determined. This could be achieved by using various existing techniques. At step 340, the error value is compared with a threshold value. The threshold value is the maximum allowable error limit in the oxygenation level due to the cross talk. If the system uses a plurality of probes, each probe may have a different threshold value. At step 350, the pulse width of the emitter drive signal is adjusted based on the error value. If the error value in the measured oxygen level is less than the threshold error value, the pulse width of the drive signal may be minimized. Also based on the calculated error, it may also be decided to what extent the pulse width can be minimized. At step 360, monitoring of the oxygenation level is continued using the emitter drive signal having modified pulse width, until the arterial oxygen saturation (SpO2) is determined.

In an example, the pulse width of the emitter drive signal may be adjusted in real time. From the measured oxygenation level, the error in the measurement due to the cross talk is calculated in real time and, based on the comparison result, the pulse width of the emitter drive signal is adjusted.

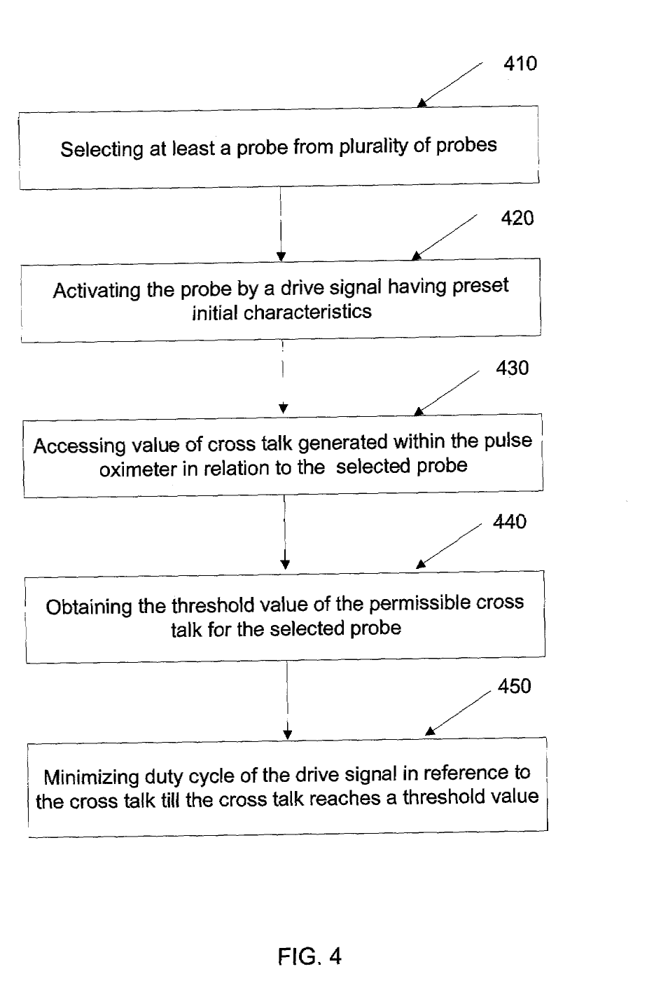

FIG. 4 is a flowchart illustrating method of configuring a pulse oximetry system for adapting multiple probes as described in an embodiment of the invention. At step 410, at least one probe is selected from a plurality of available probes. A pulse oximeter may be configured to adapt probes from different manufactures or having different configuration and specifications. The configuration of different probes and their standard values and topology may vary from each other. Plurality of probes may be connected at the same time to the pulse oximetry system or at a time one probe may be selected from the plurality of probes available. At step 420, the selected probe is activated by a drive signal. The drive signal is configured to have preset initial characteristics including a pulse width or duty cycle and a pulse repetition rate or frequency. This may be set based on the application or based on the probe. At step 430, value of cross talk generated within the pulse oximeter due to the electrical power coupling due to the cables in the pulse oximeter system. The cable may include, the cable that carries the drive signal as well. The electrical cross talk synchronous with the drive signal is measured and the value of the cross talk may be determined based on various available techniques. At step 440, a threshold value of the cross talk allowable corresponding to the selected probe is obtained. This value may be mentioned in the specification of the probe or pulse oximeter system. Alternately an error generated in the oxygenation measurement due to cross talk may be calculated and a comparison may be performed between the measured error value and a threshold error value. At step 450, the duty cycle of the detect signal may be adjusted with reference to the cross talk identified. The cross talk is compared with the threshold value corresponding to the probe and the duty cycle is adjusted till the obtained cross talk value is less than the threshold value.

If different types of cross talk, noise or any other possible errors by a probe are detected, their values may be obtained and compared individually with their corresponding threshold values. Based on the comparison result, the pulse width of the drive signal or detect signal may be adjusted accordingly.

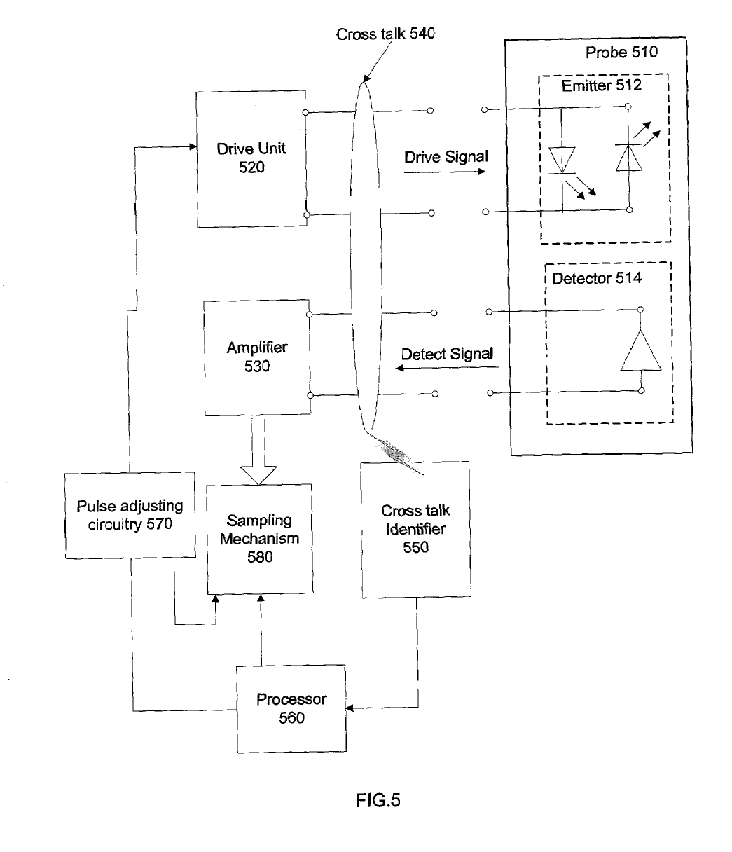

FIG. 5 is a detailed block diagram of a pulse oximeter as described in an embodiment of the invention. The pulse oximeter includes a probe 510 having an emitter 512 configured to emit light radiations. The probe 510 is connected to the patient's finger or ear lob. In an example, two LEDs are provided to emit light with two different wavelengths. The light is passed through a tissue for measuring the arterial oxygen saturation (SpO2). The probe 510 further includes a detector, generally a photo diode detector 514 configured to detect the light emitted through the tissue. A drive unit 520 is configured to activate the LEDs. The drive unit 520 generates pulses at a fixed pulse width and pulse rate, and the pulses trigger the LEDs. The magnitude of the pulses may vary based on the application. The pulse rate or the frequency of the pulses is also set initially. The drive unit 520 could be connected to the probe 510 through a cable (not shown), generally the cable is divided into two sections a longer trunk cable and a shorter probe cable. Based on the length and topology of the cable, different kinds of noise or cross talks may be introduced to the drive signal, which passes from drive unit 520 to the probe 510 through the cable.

The photo detector 514 detects the light that comes out of the tissue and the signal detected by the detector 514, the detect signal, is passed to an amplifier 530 for amplifying the detect signal. A cable carries the detect signal to the amplifier 530. There exist a resistive, inductive, capacitive cross talk (illustrated in FIG. 5 using reference numeral 540) between the drive signal and the detector signal. This cross talk 540 occurs mainly due to power coupling between the two signals. A cross talk identifier 550 is provided to measure the crosstalk 540. The cross talk identifier 550 measures the cross talk 540 generated within the pulse oximeter, the electrical cross talk synchronous with the drive signal is being measured. The cross talk 540 measured by the cross talk identifier 550 is fed to a processor 560. The processor 560 is also provided with initial values of pulse width, pulse frequency, threshold value etc. The processor 560 compares the measured value with a threshold value. The threshold value of a selected probe may be identified based on the type or nature of the probe or an operator may input the threshold value to the processor 560. The processor 560 may be further associated with pulse adjusting circuitry 570. The pulse adjusting circuitry 570 provides control signals to the drive unit 520, and is configured to adjust the pulse width of the drive signal and the detect signal. The pulse adjusting circuitry 570 adjusts the pulse width based on the instructions from the processor 560. In an example, the pulse adjusting circuitry 570 may be a part of the processor 560 itself. Thus based on the identified or measured cross talk value, the pulse adjusting circuitry 570 adjusts the pulse width, until the cross talk reaches the threshold value.

The amplifier 530 feeds the detect signal to a sampling mechanism 580, wherein the detect signal is sampled to identify the arterial oxygen saturation (SpO2). In an example, sampling of the detect signal is delayed based on the cross talk. The duty cycle of the detect signal may be increased or the sampling of the detect signal may be delayed so that the cross talk can die off or dissipate before sampling the detect signal.

The advantages of various embodiments of the invention include improving the performance of a pulse oximeter. The effect of cross talk in the arterial oxygen saturation (SpO2) measurement can be reduced or minimized. Also embodiments of the invention suggest a method of reducing power consumption of the pulse oximeters.

Thus various embodiments of the invention describe a method and system for reducing the effect of cross talk in a pulse oximeter. Another aspect of the invention suggests a method of reducing the power consumption of pulse oximeters.

As used herein, an element or step recited in the singular and proceeded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Exemplary embodiments are described above in detail. The assemblies and methods are not limited to the specific embodiments described herein, but rather, components of each assembly and/or method may be utilized independently and separately from other components described herein. Further the steps involved in the workflow need not follow the sequence in which there are illustrated in figures and all the steps in the work flow need not be performed necessarily to complete the method.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method for reducing the effect of cross talk in a pulse oximeter, comprising:
setting an initial pulse width and pulse rate for an LED drive signal;
measuring a cross talk generated within the pulse oximeter;
minimizing the pulse width of the LED drive signal with reference to the cross talk.

2. A method as in claim 1, wherein the step of measuring the cross talk includes: measuring electrical cross-talk synchronous with the LED drive signal

3. A method as in claim 2, wherein the step of measuring the cross talk includes: measuring the electrical cross talk resulting from at least one of capacitive, inductive and resistive power coupled within the pulse oximeter.

4. A method as in claim 1, wherein the step of measuring the cross talk includes: estimating an error value in a measurement of arterial oxygen saturation (SpO2).

5. A method as in claim 1, wherein the step of minimizing the pulse width includes: minimizing the pulse width until the cross talk reaches a threshold value, defined based on an SpO2 accuracy specification.

6. A method as in claim 1, wherein the step of minimizing the pulse width comprises: adjusting the pulse rate of the LED drive signal with reference to the cross talk.

7. A method as in claim 1, further comprising: reducing power consumption in the pulse oximeter by generating a modulated LED drive signal, the modulated LED drive signal being modulated based on the cross talk.

8. A method as in claim 7, wherein the method further comprises: modifying the pulse width of the LED drive signal while monitoring SpO2.

9. A pulse oximetry method comprising the steps of:
setting initial pulse width and pulse rate for an LED drive signal;
obtaining cross talk generated within a pulse oximeter;
comparing value of the cross talk with a threshold value;
adjusting the duty cycle of the LED drive signal if the cross talk value is below the threshold value; and
initiating the measurement of oxygenation once the pulse width is adjusted to a desirable value.

10. A method as in claim 9, wherein the step of comparing value of the cross talk with the threshold value comprises: comparing the cross talk value of each probe with the corresponding threshold value.

11. A method as in claim 9, wherein the step of adjusting the duty cycle of the LED drive signal comprises: adjusting the duty cycle and pulse repetition rate of the LED drive signal corresponding to each probe.

12. A method of monitoring arterial oxygen saturation using a plurality of light emitters and at least one detector, comprising the steps of:
initiating monitoring of oxygen level using an emitter drive signal having preset initial characteristics;
obtaining cross talk generated within a pulse oximeter;
determining an error value generated in the monitored oxygen level due to the cross talk;
comparing the error value with a threshold value;
adjusting the duty cycle of the emitter drive signal until the error value is below the threshold value; and
identifying the arterial oxygen saturation.

13. A method as in claim 12, wherein the step of obtaining the cross talk includes: obtaining electrical cross talk synchronous with the emitter drive signal.

14. A method as in claim 12, wherein the step of adjusting the duty cycle further comprises: adjusting sampling moments of a detect signal based on the error value such that cross talk dies off from the detector signal before sampling.

15. A method of configuring a pulse oximeter to adapt multiple probes comprising:
selecting at least one probe from a plurality of probes,
activating the selected probe by a drive signal having preset initial characteristics;
accessing value of cross talk generated within the pulse oximeter in relation to the selected probe;
obtaining a threshold value of permissible cross talk for the selected probe; and
minimizing duty cycle of the drive signal in reference to the cross talk generated due to the probe, the duty cycle being minimized until the cross talk reaches the threshold value.

16. A method as in claim 15 further comprising: identifying the selected probe and selecting a corresponding threshold value for the selected probe.

17. A pulse oximeter comprising:
at least one probe including at least two emitters configured to emit radiations in different wavelengths and at least one detector to receive radiations;
a drive unit for providing drive signal having preset initial characteristics to the emitters;
a cross talk identifier configured to identify electrical cross talk synchronous with the drive signal, generated within the pulse oximeter; and
a processor configured to adjust duty cycle of the drive signal in reference to the cross talk.

18. A pulse oximeter as in claim 17, wherein the preset initial characteristics of the drive signal include fixed initial duty cycle and pulse rate of the drive signal.

19. A pulse oximeter as in claim 17, wherein the processor is configured to adjust the duty cycle of the drive signal until the cross talk reaches a threshold value.

20. A pulse oximeter as in claim 19, wherein the threshold value is determined based on a standard value specified for each probe.

21. A pulse oximeter as in claim 17, wherein the cross talk detector detects the cross talk generated from at least one of capacitive, inductive, resistive power coupled within the pulse oximeter.

22. A pulse oximeter as in claim 17, wherein desired duty cycle for the drive signal is set prior to initializing operation of the pulse oximeter.

23. A pulse oximeter as in claim 17, wherein desired duty cycle is set for the driving signal, while the pulse oximeter is in operation.

24. A pulse oximeter as in claim 17, wherein the processor is further configured to adjust the duty cycle of detect signal, generated by the detector.
